(19) United States
(12) Patent Application Publication
(10) Pub. No.: US 2013/0331679 A1
(43) Pub. Date: Dec. 12, 2013

(54) CONTACTLESS TONOMETER

(71) Applicant: Canon Kabushiki Kaisha, Tokyo (JP)

(72) Inventor: Yasuhiro Dobashi, Matsudo-shi (JP)

(21) Appl. No.: 13/908,150

(22) Filed: Jun. 3, 2013

(30) Foreign Application Priority Data
Jun. 8, 2012 (JP) 2012-130586
Mar. 29, 2013 (JP) 2013-073218

(51) Int. Cl. A61B 3/16

(52) U.S. Cl. CPC A61B 3/165
USPC 600/401

(57) ABSTRACT

In a contactless tonometer having a mechanism of puffing compressed air by moving a piston in a cylinder, puffing of unnecessary air against the eye to be inspected is suppressed. An apparatus includes a corneal shape changing unit configured to change a shape of a cornea of an eye to be inspected by compressing air in a cylinder by using a piston, and puffing the compressed air from the nozzle to the cornea, a piston control unit configured to control operation of the piston, and an intraocular pressure measurement unit configured to measure an intraocular pressure of the eye by detecting a state of a changed shape of the cornea. This apparatus includes a piston volume changing unit configured to change an initial volume when the piston compresses the air in the cylinder.

### BACKGROUND OF THE INVENTION

1. Field of the Invention

The present invention relates to a contactless tonometer which calculates an intraocular pressure value from a corneal shape changing signal obtained by an optical detection unit when the corneal shape is changed by puffing air against the eye to be inspected.

2. Description of the Related Art

A contactless tonometer is typified by an air-puff tonometer developed by Bernard Grolman. This tonometer optically detects the applanation of the cornea upon puffing air against the cornea of the eye to be inspected from a nozzle about 11 mm apart from the cornea. A contact-Goldman type tonometer then calculates an intraocular pressure value by calibrating the time up to the applanation. Many tonometers of this type use a mechanism of compressing air in a cylinder by moving a piston inside the cylinder connected to an air-puff nozzle portion and puffing air from a nozzle. In addition, a solenoid is generally used for a drive mechanism for the piston because the solenoid has a high initial torque and a simple arrangement.

In addition, a contactless tonometer is required to have a wide measurement range from a low intraocular pressure to a high intraocular pressure for a disease such as glaucoma. In order to measure a high intraocular pressure, it is necessary to puff sufficient air against the eye to be inspected. A cylinder volume is designed with reference to a high intraocular pressure. For this reason, for the eye to be inspected which has a low intraocular pressure, the amount of air puffed is adjusted by changing a drive current or drive time for the solenoid in accordance with the intraocular pressure value of the eye.

A mechanism using a solenoid is inexpensive and has a simple arrangement but is known to have several demerits. The solenoid has a simple structure constituted by only a winding and a permanent magnet and is configured to operate in only one direction. This mechanism, therefore, needs to use a return mechanism using a return spring and the like. In general, the actuating force of the solenoid is sufficiently larger than that of the return spring. Once the solenoid is energized to drive the piston, the inertia force due to the weight of the piston acts on the piston even after the interruption of a current. This makes it difficult to stop the piston at a target position.

When measuring the eye to be inspected which has a low intraocular pressure, the amount of air required for applanation is small, and it is necessary to stop the piston at a considerably early stage with respect to the piston drive range in the cylinder. However, unnecessary air is puffed against the eye due to the inertia force of the piston. This makes the object feel uncomfortable.

As an invention for solving the above problem, for example, 1) there is known the invention disclosed in Japanese Patent Application Laid-Open No. H09-201335, which decreases the moving amount of the piston due to its inertia force after the interruption of a piston drive current by increasing the drive voltage applied to the solenoid for driving the piston at a gradual rise rate.

In addition, 2) there is known the mechanism disclosed in Japanese Patent Application Laid-Open No. 2002-034927, which lets air escape through an electromagnetic valve to prevent compressed air in the cylinder from being puffed against the eye to be inspected. This invention has the mechanism for letting air escapes from the cylinder through the electromagnetic valve, and is configured to open the electromagnetic valve at a proper timing to reduce unnecessary air puffed against the eye to be inspected by predicting the timing of opening the electromagnetic valve from the first measurement in consideration of the response delay characteristic of the electromagnetic valve.

Even in a circuit configured to gradually increase the rise rate of an applied voltage as in the arrangement disclosed in Japanese Patent Application Laid-Open No. H09-201335, it is not possible to prevent air from being puffed due to the inertial force of the piston, and a complicated control circuit is required for variable applied voltages.

Furthermore, even if it is possible to suddenly stop the piston by using some kind of control mechanism, the air compressed in the cylinder leaks out from a puffing nozzle because the air is higher in pressure than the atmospheric pressure. This invention does not lead to the solution of the fundamental problem that uncomfortable air is puffed against an object.

The method of letting compressed air in the cylinder escape by opening the electromagnetic valve, which is disclosed in Japanese Patent Application Laid-Open No. 2002-034927, is theoretically effective. However, in order to instantaneously release the air compressed in the cylinder, the opening of the electromagnetic valve needs to be sufficiently large as compared with the nozzle. That is, a large electromagnetic valve is required. A large electromagnetic valve costs high and is difficult to mount in a limited space in the apparatus. This raises the hurdle for the use of the above method.

### SUMMARY OF THE INVENTION

The present invention provides a contactless tonometer which can solve the above problem and suppress the puffing of unnecessary air against the eye to be inspected with a low-cost, simple arrangement.

According to an aspect of the present invention, there is provided a contactless tonometer including a corneal shape changing unit configured to change a shape of a cornea of an eye to be inspected by compressing air in a cylinder by using a piston which is disposed in the cylinder and operates from a movement start position, and puffing the compressed air from inside of the cylinder to the cornea, a piston control unit configured to control operation of the piston, and an intraocular pressure measurement unit configured to measure an intraocular pressure of the eye by detecting a state of a changed shape of the cornea, comprising a piston volume changing unit configured to change an initial volume when the piston compresses the air in the cylinder.

The contactless tonometer according to the present invention can puff optimal air in accordance with an intraocular pressure value by changing the drive position of the piston relative to the cylinder. In addition, when controlling the piston by driving the solenoid, it is possible to prevent the puffing of air unnecessary for measurement due to the inertia force of the piston.

In addition, it is possible to provide an inexpensive, compact apparatus because it can be formed by only adding a piston position detection mechanism to a conventional apparatus.

Further features of the present invention will become apparent from the following description of exemplary embodiments with reference to the attached drawings.

### BRIEF DESCRIPTION OF THE DRAWINGS

FIG. 1 is a view showing the outer appearance of a contactless tonometer.

FIG. 2 is a view showing the arrangement of the optical system of a measurement portion.

FIG. 3 is a block diagram of a system according to the first embodiment.

FIGS. 4A, 4B and 4C are views for explaining piston positions in a conventional control method.

FIG. 5 is a graph showing the relationship between a corneal shape changing signal and a pressure signal in the conventional control method.

FIGS. 6A, 6B and 6C are views for explaining piston positions in a control method according to the first embodiment.

FIG. 7 is a graph showing the relationship between a corneal shape changing signal and a pressure signal in a control method according to the first embodiment.

FIG. 8 is a flowchart for explaining the embodiment.

FIGS. 9A and 9B are views showing a piston structure in the second embodiment.

FIGS. 10A, 10B, 10C and 10D are views for explaining piston states and positions in the second embodiment.

FIG. 11 is a graph showing the relationship between spring elastic force and piston drive force in the second embodiment.

FIG. 12 is a graph showing the relationship between a corneal shape changing signal and a pressure signal in a piston structure in the second embodiment.

### DESCRIPTION OF THE EMBODIMENTS

Preferred embodiments of the present invention will now be described in detail in accordance with the accompanying drawings.

### First Embodiment

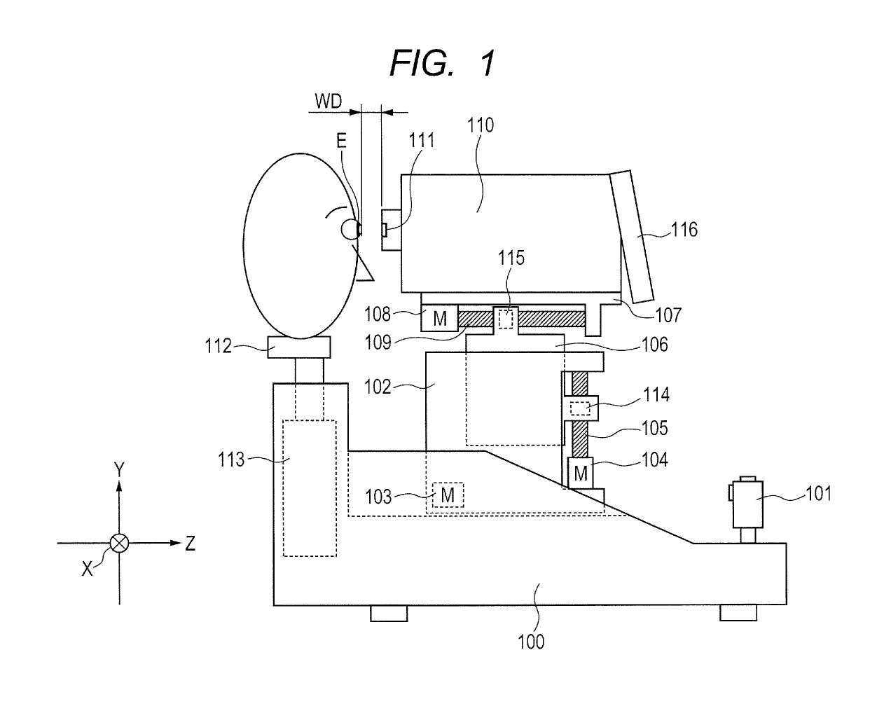

FIG. 1 is a view showing the schematic arrangement of a contactless tonometer according to the present invention.

A frame 102 can move in the horizontal direction (to be referred to as an X axis direction hereinafter) relative to a base 100. A drive mechanism in the X axis direction includes an X axis motor 103 fixed on the base 100, a feed screw (not shown) coupled to a motor output shaft, and a nut (not shown) fixed to the frame 102 so as to be movable on the feed screw in the X axis direction. The motor 103 rotates to move the frame 102 in the X axis direction through the feed screw and the nut.

A frame 106 can move in the vertical direction (to be referred to as the Y axis direction hereinafter) relative to the frame 102. A drive mechanism in the Y axis direction includes a Y axis motor 104 fixed on the frame 102, a feed screw 105 coupled to the motor output shaft, and a nut 114 fixed on the frame 106 so as to be movable on the feed screw in the Y axis direction. The motor 104 rotates to move the frame 106 in the Y axis direction through the feed screw and the nut.

A frame 107 can move in the back-and-forth direction (to be referred to as the Z axis direction hereinafter) relative to the frame 106. A drive mechanism in the Z axis direction includes a Z axis motor 108 fixed on the frame 107, a feed screw 109 coupled to the motor output shaft, and a nut 115 fixed on the frame 106 so as to be movable on the feed screw in the Z axis direction. The motor 108 rotates to move the frame 107 in the Z axis direction through the feed screw and the nut.

In this case, the movement of the frame 102 in the X axis direction, the movement of the frame 106 in the Y axis direction, and the movement of the frame 107 in the Z axis direction respectively correspond to the movements of the tonometer in the horizontal direction, the vertical direction, and the back-and-forth direction, which corresponds to the direction to approach and separate from the object, relative to the object.

A measurement portion 110 is fixed on the frame 107 to perform measurement. The object-side end portion of the measurement portion 110 is provided with a nozzle 111 for discharging air required for intraocular pressure measurement. The object-side end portion of the measurement portion 110 is provided with an LCD monitor 116 as a display member for observing an eye E to be inspected.

The base 100 is provided with a joy stick 101 as an operation member for positioning the measurement portion 110 relative to the eye E.

When performing intraocular pressure measurement, the object rests his/her chin on a chin rest 112 and presses his/her forehead against the forehead rest portion of the face rest frame (not shown) fixed on the base 100, thereby fixing the position of the eye to be inspected. A chin rest motor 113 can adjust the chin rest 112 in the Y-axis direction in accordance with the size of the face of the object.

FIG. 2 shows the arrangement of an optical system in the measurement portion 110. A nozzle 22 is disposed on the central axis of a plane parallel glass 20 and objective lens 21 so as to face a cornea Ec of the eye E, and an air chamber 23, an observation window 24, a dichroic mirror 25, a prism stop 26, an imaging lens 27, and a CCD 28 are sequentially arranged behind the nozzle 22. These components constitute the light receiving optical path and alignment detection optical path of the observation optical system for the eye E.

An objective mirror barrel 29 supports the plane parallel glass 20 and the objective lens 21. Anterior ocular illumination light sources 30a and 30b for illuminating the eye E are arranged outside the objective mirror barrel 29.

For the sake of simplification, FIG. 2 shows the anterior ocular illumination light sources 30a and 30b arranged in the vertical direction. In practice, however, they are arranged to face the optical axis in the direction perpendicular to the drawing surface.

A relay lens 31, a half mirror 32, an aperture 33, and a light receiving element 34 are arranged in the reflecting direction of the dichroic mirror 25. Note that the aperture 33 is disposed at a position where it becomes conjugate to a cornea reflection image of the measurement light source 37 (to be described later) when the corneal shape changes into a predetermined shape. The aperture 33 and the light receiving element 34 constitute a shape change detection light receiving optical system when the shape of the cornea Ec changes in a visual axis direction.

The relay lens 31 is designed to form a cornea reflection image having a size almost equal to that of the aperture 33 when the cornea Ec changes into a predetermined shape.

A half mirror 35, a projection lens 36, and a measurement light source 37 formed from a near infrared LED with an invisible light wavelength, which also serves for alignment, for the eye E to be measured and inspected are arranged in the incident direction of the half mirror 32. A fixation target light source 38 formed from an LED for the visual fixation of the object is disposed in the incident direction of the half mirror 35.

A pressure sensor 45 for monitoring the internal pressure of the air chamber and a transfer tube 44 for transferring compressed air from a cylinder 43 are connected to the inside of the air chamber 23. The transfer tube may have any form. For example, this tube may be a bellows tube like that shown in FIG. 2 or a metal tube. Alternatively, the cylinder 43 may be directly connected to the air chamber 23 without using the transfer tube 44. A piston 40 is fitted in the cylinder 43. A solenoid 42 drives the piston 40. A drive lever 41 connected to the solenoid 42 and the piston 40 converts the rotary movement of the solenoid 42 into the linear movement of the piston 40. As the piston 40 moves in the cylinder 43 at high speed, the compressed air in the cylinder 43 is sent to the air chamber 23 and is puffed against the eye E through the nozzle 22. In the present invention, the arrangement constituted by the cylinder 43, the piston 40, and the like form an example of a corneal shape changing unit which compresses air in the cylinder by using the piston which is disposed in the cylinder and moves from the movement start position, and changes the shape of the cornea by puffing the compressed air against the cornea of the eye to be inspected from the inside of the cylinder.

A sensor dog 46 for detecting a piston position is connected to the piston 40 to practice the present invention. It is possible to detect the position of the piston 40 by using the sensor dog 46 and a detection switch 47.

In this case, the detection switch 47 may have any form as long as it can detect the position of the piston 40. For example, this switch may be a photointerrupter, microswitch, or potentiosensor. In addition, the sensor dog 46 and the detection switch 47 need not be arranged near the cylinder 43 as shown in FIG. 2 and can be arranged near the solenoid 42 to detect the position of the piston 40 from the rotational angle of the solenoid 42. The present invention has exemplified these arrangements as an example of a piston position detection unit which detects the position of the piston.

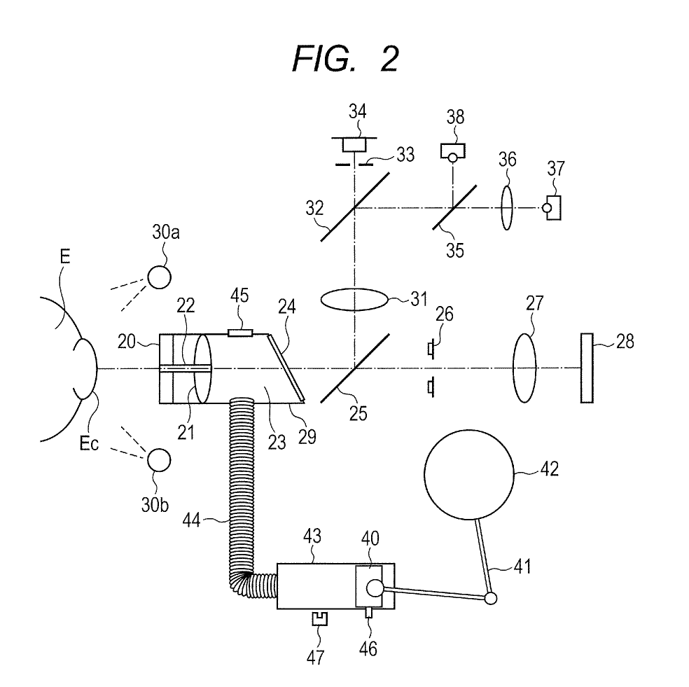

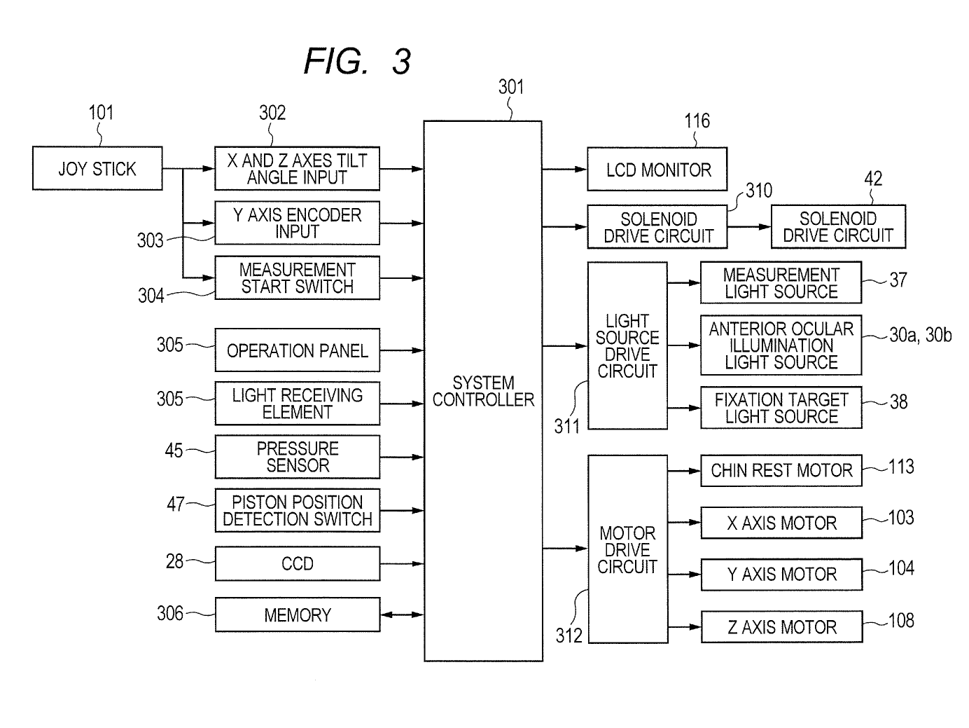

FIG. 3 is a system block diagram. A system controller 301 which controls the overall system includes a program storage unit, a data storage unit storing data for correcting an intraocular pressure value, an input/output controller which controls input/output operation with respect to various types of devices, and a computation processing unit which computes data obtained from various types of devices.

An X and Z axes tilt angle input 302 obtained when the operator tilts the joy stick 101, which positions the measurement portion 110 to the eye E and causes the measurement portion 110 to start measurement, back and forth and left and right, a Y axis encoder input 303 obtained when the operator rotates the joy stick 101, and an input from a measurement start switch 304 at the time of pressing a measurement start button are connected from the joy stick 101 to the system controller.

A print button, chin rest up and down buttons, and the like are arranged on an operation panel 305 on the base 100 (not shown). When the operator performs button input operation, the panel notifies the system controller of a corresponding signal.

A memory 306 stores the anterior ocular segment image of the eye E captured by the CCD 28. Alignment detection is performed by extracting the reflection images of the pupil and cornea of the eye E from the image stored in the memory 306. The anterior ocular segment image of the eye E captured by the CCD 28 is combined with character and graphic data to display the anterior ocular segment image, measurement values, and the like on the LCD monitor 116.

The memory 306 stores the corneal shape changing signal received by the light receiving element 34 and a signal from the pressure sensor 45 disposed in the air chamber 23. An arrangement including the light receiving element 34 and configured to measure the intraocular pressure of an eye to be inspected by detecting a corneal shape changing signal representing how the corneal shape has changed is described as an example of an arrangement functioning as an intraocular pressure measurement unit in the present invention.

The X axis motor 103, the Y axis motor 104, the Z axis motor 108, and the chin rest motor 113 are driven by commands from the system controller 301 via a motor drive circuit 312. The measurement light source 37, the anterior ocular illumination light sources 30a and 30b, and the fixation target light source 38 are controlled to turn on/off and change their amounts of light by commands from the system controller 301 via a light source drive circuit 311.

The solenoid 42 is controlled by signals from the system controller 301. The system controller 301 changes a drive current and turns on/off the application of a voltage to the solenoid 42 via a solenoid drive circuit 310.

In this embodiment, a rotary solenoid is used for the solenoid 42. This rotatory solenoid is designed such that a movable pin moves in a coil wound by a copper wire upon application of a voltage, and mechanical components such as a bearing convert linear movement in to rotary movement in addition, since the rotary torque is limited in a unique direction, the solenoid is structured to return to the initial position by a built in coil spring.

Setting the value of the drive current flowing in the solenoid 42 high under the control of the solenoid drive circuit 310 will generate high torque in the solenoid 42. This can rotate the solenoid at high speed. In addition, the rotatory solenoid incorporates the coil spring to return to the initial position. This makes it possible to move and hold the solenoid 42 to an arbitrary angle by controlling a current value while balancing with the coil spring by supplying a minute current to the solenoid 42. Note that the arrangement including the solenoid drive circuit 310 and configured to operate the piston 40 is an example of a piston controller for controlling the operation of the piston in the present invention. That is, in this example, the solenoid operates the piston, and the piston controller controls the piston by variably controlling a drive current for the solenoid and performing ON/OFF control.

The effects of the present invention will be described next by comparing the first embodiment with the conventional control method, that is, the case in which the movement start position of the piston 40 is set at the distal end portion of the cylinder 43.

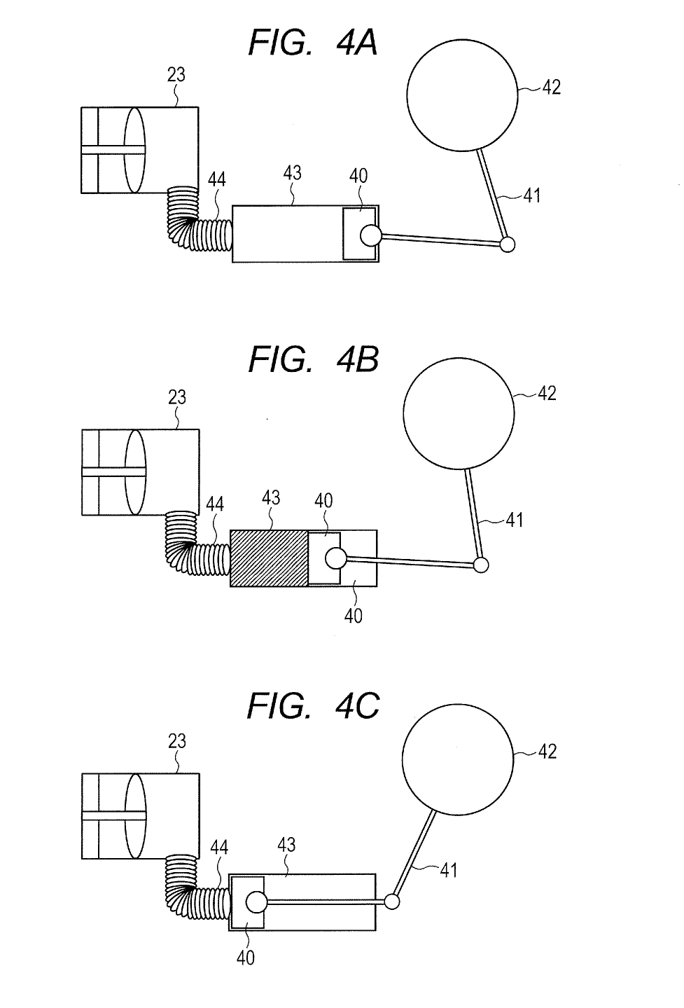

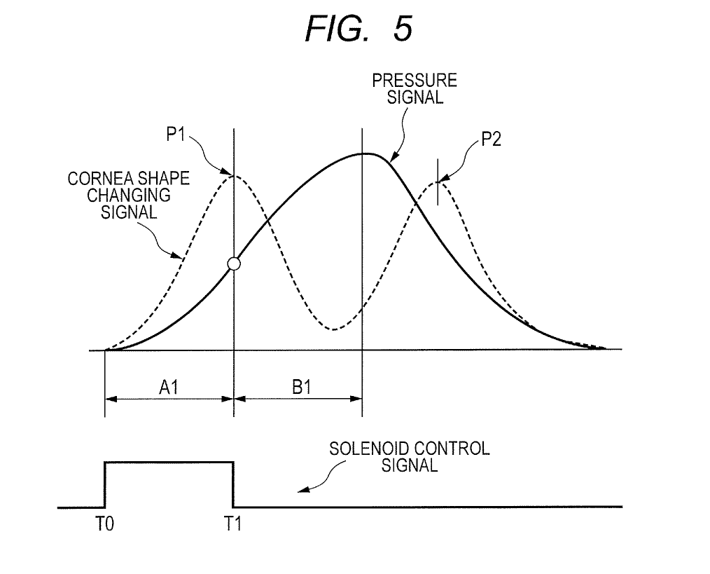

Solenoid control by the system controller 301 at the time of intraocular pressure measurement in the related art will be described first with reference to FIGS. 4A to 5. FIGS. 4A to 4C each show only the air puffing unit extracted from the optical arrangement shown in FIG. 2. Each of FIGS. 4A to 4C is a view showing the energized state of the solenoid 42 and the corresponding position of the piston 40. For the sake of ease of explanation, a description of the sensor dog 46 and detection switch 47 which are not necessary in the related art will be omitted. FIG. 5 shows the relationship between a solenoid control signal, a pressure signal in the air chamber 23 which is obtained by the pressure sensor 45 at the corresponding time of intraocular pressure measurement, and the changed shape state (to be referred to as a corneal shape changing signal hereinafter) of the eye E which is detected by the light receiving element 34. Referring to FIG. 5, the abscissa represents the time from the start time of measurement, and the ordinate represents the level of each signal.

In addition, a time period A1 shown in FIG. 5 indicates the time period from the start of detection of a pressure signal and a corneal shape changing signal to a maximum value P1 of the corneal shape changing signal. This time period corresponds to the state change from FIGS. 4A to 4B. Likewise, a time period B1 in FIG. 5 corresponds to a state in which a drive current to the solenoid 42 is interrupted. This time period corresponds to the state change from FIGS. 4B to 4C. The solenoid control signal in FIG. 5 indicates the energization period of the solenoid from T0 to T1. In the first embodiment, the energization period of the solenoid coincides with the time period A1.

FIG. 4A shows the piston position immediately before the energization of the solenoid 42. The piston 40 is fixed to the start end point of the cylinder as the initial position by the torque of the coil spring incorporated in the solenoid 42. When the apparatus completes alignment with the eye to be inspected and starts intraocular pressure measurement, the system controller 301 drives the solenoid 42 at high speed to compress air in the air chamber 23 by the piston 40 pushed up by the solenoid 42. As the internal pressure of the air chamber 23 rises, air is puffed from the nozzle 22 against the cornea Ec of the eye E to start applanation.

As described above, the amount of light entering the light receiving element 34 is designed to be maximum at the instant when the cornea Ec is applanated by puffed air. The point P1 at which the corneal shape changing signal is maximized in FIG. 5 indicates the instant at which the cornea Ec changes from a convex state to a concave state. Upon detecting the maximum value of this corneal shape changing signal, the system controller 301 stops a drive current to the solenoid 42 and calculates the intraocular pressure value of the eye E from the simultaneously input pressure signal value indicated by the circle in FIG. 5.

The intraocular pressure values of healthy eyes generally range from 10 to 20 mmHg, and it is known that the intraocular pressure of an eye with an eye disease such as glaucoma has a high intraocular pressure value equal to or more than 20 mmHg. For this reason, the apparatus is required to have a wide measurement range from about 0 to mmHg, and the volume of the cylinder 43 and the acceleration speed of the piston 40 are designed to enable measurement of the maximum intraocular pressure value. In other words, the cylinder volume of the apparatus is too large for an eye to be inspected which has a general intraocular pressure value equal to or less than the maximum intraocular pressure value.

In conventional measurement, therefore, the apparatus has performed control to reduce unnecessary air puffed against the eye to be inspected by reducing the drive current to the solenoid 42 and quickening the timing of drive current interruption.

It is however known that the piston 40 has inertia force due to its own weight and keeps moving after the interruption of a drive current to the solenoid 42.

FIG. 4B shows the position of the piston 40 at the instant when the point P1 in FIG. 5 is detected. FIG. 4C shows the position where the piston 40 has finally stopped. Even after a drive current is interrupted, the piston 40 moves from the position in FIG. 4B to the position in FIG. 4C while keeping almost the same velocity to compress the residual air in the cylinder 43 which is indicated by the hatching in FIG. 4B. As a result, the compressed air is puffed as air unnecessary for measurement against the eye to be inspected. The time period B1 shown in FIG. 5 indicates the relationship between a corneal shape changing signal and a pressure signal when the piston 40 moves due to inertial force. It is known that even after the drive current to the solenoid 42 is interrupted, the piston 40 keeps compressing the air in the cylinder 43 to keep increasing the pressure in the the air chamber 23. As a result, the air puffed from the nozzle 22 changes the state of the cornea Ec from an applanation state to a concave state. This decreases a corneal shape changing signal value.

After the piston 40 stops in the state in FIG. 4C, the torque of the coil spring incorporated in the solenoid 42 acts to return the piston to the start end point of the cylinder which is the initial position shown in FIG. 4A.

Note that stopping puffing air will return the state of the cornea Ec from a concave state to a normal convex state through an applanation state. At this time, a corneal shape changing signal has a second peak point P2 as shown in FIG. 5.

This embodiment has exemplified the case in which the apparatus interrupts a drive current to the solenoid 42 upon detecting the maximum value of a corneal shape changing signal because the timing of drive current interruption is not important. Although a detailed description will be omitted, if it is possible to detect a peak value of a corneal shape changing signal, the apparatus may interrupt a drive current at the instant when, for example, a corneal shape changing signal or a pressure signal exceeds a predetermined threshold.

As already described above, since the cylinder 43 of the conventional contactless tonometer is designed with reference to the maximum intraocular pressure, there is the problem that air unnecessary for measurement is puffed against the eye to be inspected due to the inertia force of the piston 40. The present invention therefore solves the above problem by changing the movement start position of the piston 40 and changing (reducing) the initial volume of the cylinder 43.

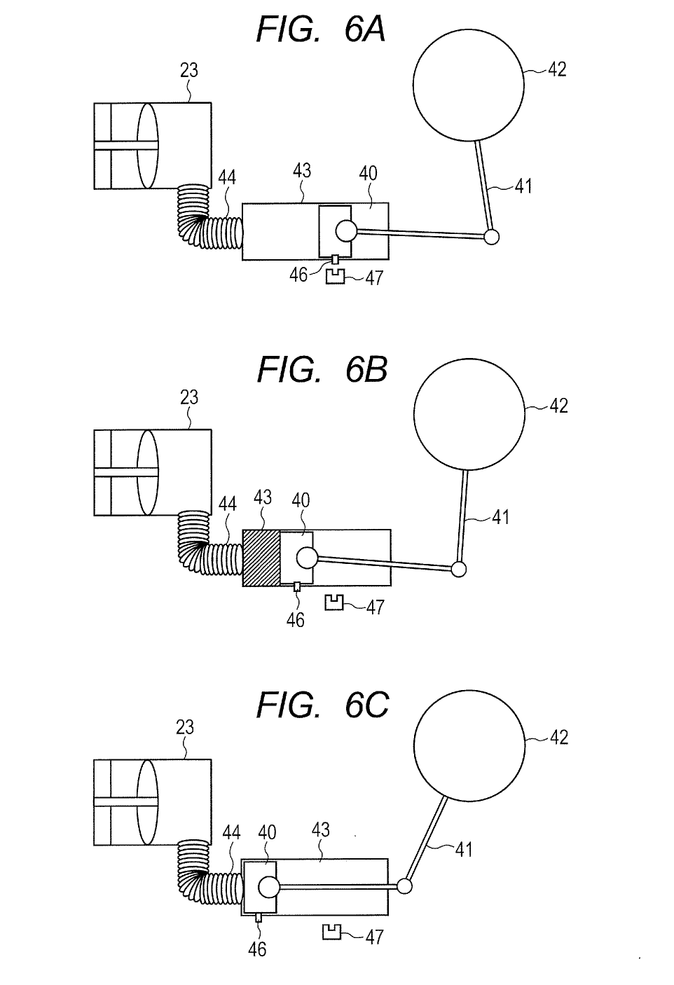

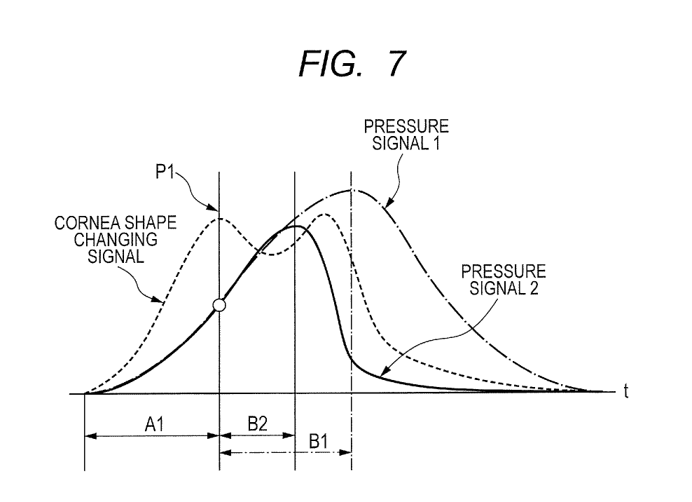

The first embodiment will be described in detail next with reference to FIGS. 6A to 7.

Each of FIGS. 6A to 6C shows only the air puffing unit extracted from the optical arrangement shown in FIG. 2. Each of FIGS. 6A to 6C is a view showing the energized state of the solenoid 42 and the corresponding position of the piston 40. FIG. 7 shows the relationship between the pressure signal in the air chamber 23 which is obtained by the pressure sensor 45 at the time of intraocular pressure measurement and the corneal shape changing signal detected by the light receiving element 34. The abscissa represents the time elapsed since the start time of measurement, and the ordinate represents the level of each signal. As in FIG. 5, the dotted line represents the corneal shape changing signal, and the solid line represents the pressure signal (pressure signal 2). For comparison, the chain line represents a pressure signal (pressure signal 1) in the conventional control method. As described above, in the first embodiment, since an energization period of the solenoid coincides with time period A1 described above, a description about solenoid control will be omitted.

FIG. 6A shows the movement start position of the piston 40 in the present invention. In this case, the sensor dog 46 and detection switch 47 described above are added to the arrangement shown in FIG. 4A, and the position where the detection switch 47 detects the sensor dog 46 is set as the movement start position of the piston 40. The relative positions of the sensor dog 46 and detection switch 47 for detecting the movement start position of the piston 40 are set to optimal positions required to obtain an arbitrary maximum intraocular pressure value. For example, it is possible to easily calculate the volume of the cylinder 43 which is required to measure the eye to be inspected which has a maximum intraocular pressure of 30 mmHg. Setting the detection switch 47 at the position where the calculated cylinder volume is obtained makes it possible to form a measurement system with a maximum intraocular pressure of 30 mmHg being an upper limit.

Upon starting measurement, the apparatus energizes the solenoid 42 to drive the piston at high speed in a time interval A1 in FIG. 7 as in conventional control operation. As the piston 40 moves in the cylinder 43 at high speed, the pressure signal in the air chamber 23 rises, and air puffing from the nozzle 22 will then start the applanation of the cornea Ec. As a consequence, the corneal shape changing signal begins to rise.

If the intraocular pressure value of the eye to be inspected is smaller than the maximum intraocular pressure value set by the detection switch 47, the system controller 301 detects a corneal shape changing signal peak value P1 before the piston 40 reaches the terminal end point of the cylinder 43 shown in FIG. 6C which has started from the position in FIG. 6A (FIG. 7).

Upon obtaining the corneal shape changing signal peak P1, the system controller 301 interrupts the drive current to the solenoid 42, and calculates the intraocular pressure value of the eye E from the simultaneously input pressure signal value indicated by the circle in FIG. 5.

FIG. 6B shows the position of the piston at the instant when the corneal shape changing signal peak P1 is obtained. In this case, the piston 40 keeps moving to the position in FIG. 6C, which is the terminal end point of the cylinder 43, due to inertia force even after the interruption of a drive current to the cylinder 43, as described in the conventional control operation.

However, since the movement start position of the piston 40 is changed to a forward position relative to that in the conventional control operation, the distance from the position in FIG. 6B to the position in FIG. 6C is sufficiently shorter than that in the conventional control operation. As is obvious, therefore, the amount of residual air corresponding to the hatched portion shown in FIG. 6B is sufficiently smaller than that in the conventional control operation. In addition, a time interval B2 shown in FIG. 7, which corresponds to the interval from the state in FIG. 6B to the state in FIG. 6C, that is, the time during which the piston 40 moves due to the inertia force, is shorter than the time period B1 in the conventional control operation.

As described above, changing the movement start position of the piston 40 and changing the initial volume of the cylinder 43 can suppress the puffing of unnecessary air against the eye to be inspected and puff an optimal amount of air in accordance with the intraocular pressure value of the eye.

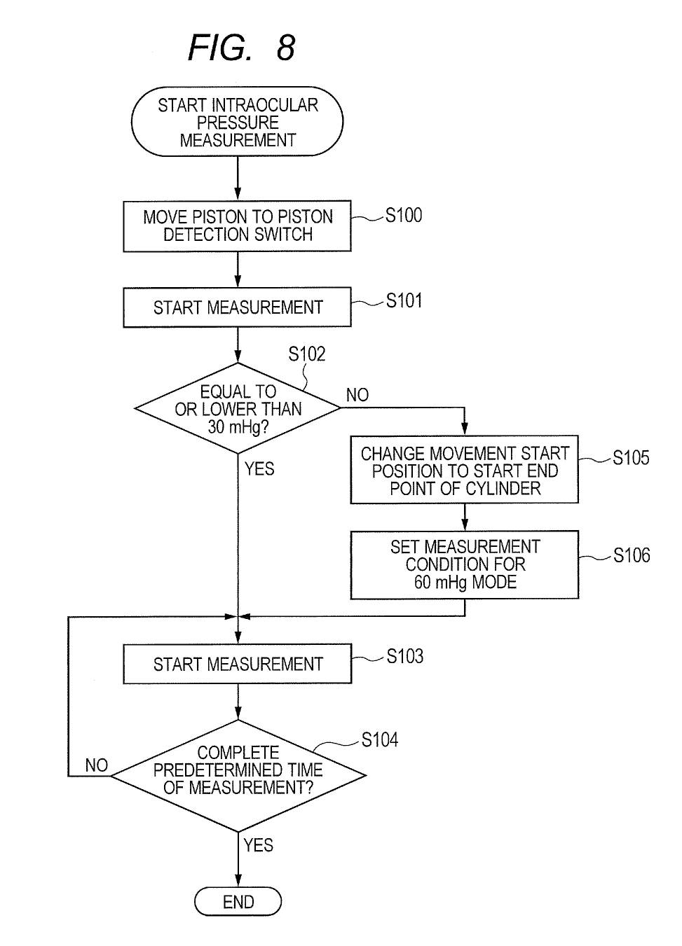

An example of the embodiment using the present invention will be finally described with reference to the flowchart of FIG. 8 for a measurement procedure.

Preparation before the start of measurement will be briefly described first. The examiner lets the object rest his/her chin on the chin rest 112, and adjusts the eye to be inspected at a predetermined height in the Y-axis direction by using the chin rest motor 113. The examiner operates the joy stick 101 up to a position where a cornea reflection image of the eye E displayed on the LCD monitor 116 is displayed, and presses the measurement start button.

When the examiner presses the measurement start button, the apparatus starts automatic alignment. At the time of alignment, the prism stop 26 splits the cornea bright spot formed by the cornea Ec, and the anterior ocular illumination light sources 30a and 30b illuminate the eye E. The resultant image of the eye E is then formed on the CCD 28, together with the bright spot images of the anterior ocular illumination light sources 30a and 30b. The system controller 301 stores the captured anterior ocular segment image of the eye E in the memory 306, and performs alignment via the motor drive circuit 312 based on the position information at each bright spot extracted from the eye E and the cornea reflection image. Upon completing the alignment, the apparatus starts measurement in the following procedure.

In step S100, the system controller 301 drives the piston 40 at low speed by supplying a minute current to the solenoid 42 to move the piston 40 to the movement start position. The movement start position of the piston has been determined by the detection result obtained by the piston position detection switch 47. Upon detecting the movement start position of the piston, the system controller 301 starts control to hold the piston 40 at the detected position while balancing with the return force of the coil spring incorporated in the solenoid 42. Assume that in this embodiment, the piston position detection switch 47 is set at the position to ensure a cylinder volume necessary for the measurement of the eye to be inspected which has a maximum intraocular pressure of 30 mmHg. The arrangement for moving the piston 40 to the movement start position and holding it at the movement start position is described as an example of a piston volume changing unit which can change the initial volume when the piston 40 compresses air in the cylinder 43 in the present invention. A piston control unit moves and holds the piston as described above based on the detection result obtained by the above piston position detection unit. The piston volume changing unit changes the initial volume by changing the movement start position of the piston 40 as described above.

Upon determining that the piston 40 has moved to the movement start position, the system controller 301 drives the piston 40 at high speed to start intraocular pressure measurement by increasing a current value supplied to the solenoid 42 in step S101. The current flowing in the solenoid 42 at this time is the value calculated from the cylinder volume determined by the movement start position of the piston and having undergone correction at the time of factory shipment to allow the measurement of 30 mmHg with the pressure of air puffed from the nozzle 22.

In step S102, the system controller 301 determines whether the measured intraocular pressure value is smaller than 30 mmHg. Since the measurement start position of the piston 40 has been changed in step S100, the apparatus according to this embodiment can measure only eyes to be inspected up to 30 mmHg. For this reason, the system controller 301 determines whether the measurement intraocular pressure value is 30 mmHg. If the measured intraocular pressure value is smaller than 30 mmHg, the process shifts to step S103. In step S103, the apparatus actually performs measurement. The process then shifts to step S104 to determine whether the apparatus has completed measurement a predetermined number of times. If the number of times of measurement has not reached the predetermined number, the process returns to step S103 to perform measurement again. If the number of times of measurement has reached the predetermined number, the apparatus terminates intraocular pressure measurement. If the predetermined number of times is determined as one, since the measurement condition in step S101 holds, the apparatus terminates the intraocular pressure measurement. Note that the apparatus can be configured such that if the apparatus determines in step S104 that further measurement is necessary, the process returns to step S102, after going through step S103 of performing measurement again, to determine whether it is necessary to change the measurement start position. If, for example, an intraocular pressure value is near 30 mmHg and increases as the apparatus further performs measurement, this arrangement can suitably correspond to such a situation. In addition, a region in the system controller 301 which functions as a determination unit determines whether the above measured intraocular pressure value is equal to or more than a predetermined value.

In this case, the movement start position of the piston at the time of measurement in step S103 differs depending on whether the 30 mmHg mode or the 60 mmHg mode is set. If the system controller 301 determines in step S102 that the intraocular pressure value of the eye to be inspected is smaller than 30 mmHg, the apparatus starts driving the piston 40 from the detection position designated in step S100.

If the system controller 301 determines in step S102 that the intraocular pressure value of the eye to be inspected is equal to or more than 30 mmHg, the apparatus performs measurement from the movement start position of the piston in the 60 mmHg mode (to be described later).

Control operation performed by the system controller 301 upon determining in step S102 that a measurement result is equal to or more than 30 mmHg will be described next. As described above, the apparatus cannot measure an intraocular pressure equal to or more than 30 mmHg at the movement start position of the piston 40 set in step S101. For this reason, the system controller 301 changes the piston start position to the drive start end point of the cylinder 43 in step S105 (step S105). For control operation in this case, the system controller 301 is only required to stop energization to the solenoid 42. This causes the piston 40 to automatically move the drive start end point of the cylinder 43 due to the return force of the coil spring of the solenoid 42. That is, if the intraocular pressure is equal to or more than a predetermined value, the piston volume changing unit increases the initial volume of the piston. Upon changing the movement start position of the piston 40, the system controller 301 starts measuring a current value for the measurement of 60 mmHg in step S106. In this case, as in the measurement of 30 mmHg, a set current value for the measurement of 60 mmHg is also a value having undergone correction at the time of factory shipment.

Upon completion of intraocular pressure measurement following the above flowchart, the system controller 301 performs control operation in accordance with a general measurement routine for switching between the left and right eyes and printing of a measurement result, thereby completing all operation.

This embodiment has exemplified the case using one detection switch. However, the apparatus can have a plurality of detection switches for 15 mmHg, 30 mmHg, 45 mmHg, and the like. It is possible to measure the eye to be inspected with a smaller optimal amount of air puffed by performing measurement with a cylinder volume for 30 mmHg in the first intraocular pressure measurement and setting the movement start position of the piston 40 in accordance with the first measurement result in the subsequent intraocular pressure measurement. If, for example, the first measurement result is 10 mmHg, it is possible to perform measurement with a more comfortable amount of air by setting the movement start position of the piston in accordance with the detection switch position for 15 mmHg. In addition, this embodiment has exemplified the case in which the apparatus starts measurement first in the 30 mmHg mode, and then performs measurement in the 60 mmHg mode in step S106, as needed. However, the apparatus may be configured to start measurement in the 60 mmHg mode and then perform measurement upon shifting to the 30 mmHg mode if the measurement value is equal to or less than 30 mmHg. That is, if the intraocular pressure measured by the intraocular pressure measurement unit is equal to or less than a predetermined value, the piston volume changing unit decreases the initial volume of the piston.

As an application example, it is possible to perform more flexible control operation by using an analog detection unit such as a potentiometer as the detection switch 47 instead of a digital detection unit. For example, it is possible to puff comfortable air against all eyes to be inspected by setting the movement start position of the piston in the second and subsequent measurement procedures to a position corresponding to the maximum intraocular pressure value which allows measurement of “first measurement result+5 mmHg”. That is, in such a case, the piston volume changing unit changes the initial volume of the piston in accordance with the intraocular pressure obtained by adding a predetermined value to a measured intraocular pressure.

### Second Embodiment

In general, in a structure in which the movement start position of a piston 40 is determined as in the case of a conventional product, a hole for air discharge is formed near the middle of a cylinder 43 for the purpose of shortening the air puffing time for the eye to be inspected.

Since the air in the cylinder 43 is not compressed until the piston 40 passes by the hole, the piston 40 increases its driving speed without any air resistance and starts compressing the air upon passing by the hole. Assume that the piston 40 is driven by the same force. In this case, if the initial velocity at the start time of air compression is high, the velocity of air puffed against the eye to inspect is accordingly high. This shortens the time to reach a pressure necessary for measurement. The arrangement according to the first embodiment has the demerit that since the movement start position of the piston 40 is set at an arbitrary position, no hole can be formed in the cylinder, and it is impossible to increase the initial velocity of the piston 40 at the start time of air compression.

Under the circumstance, the second embodiment described below is proposed, against the first embodiment, for the purpose of increasing the initial velocity of the piston 40 at the start time of air compression.

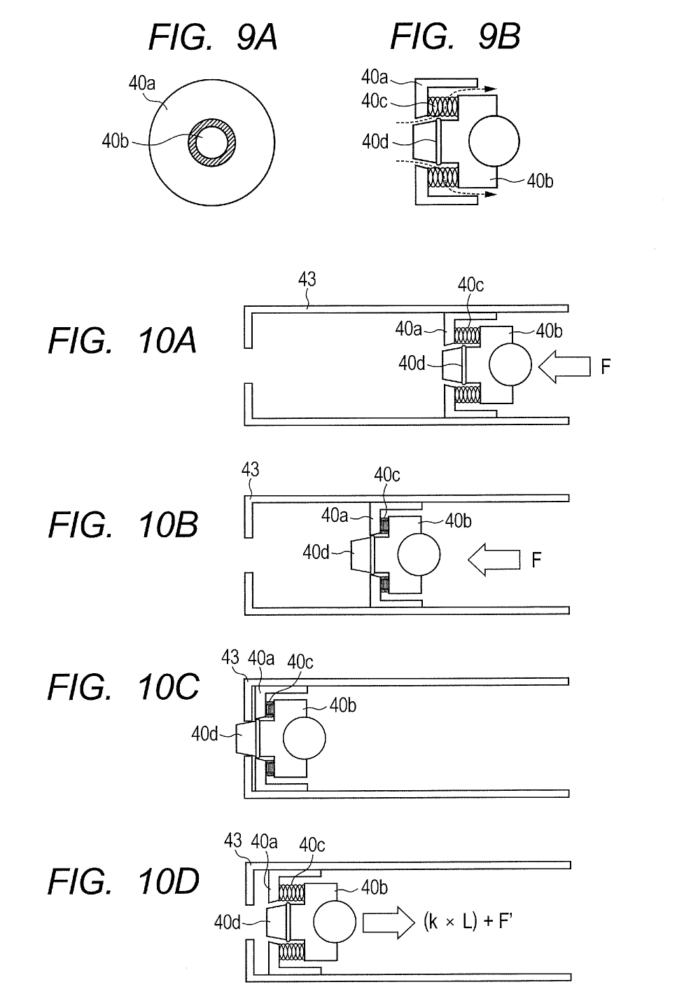

FIGS. 9A and 9B show the structure of the piston 40 which is a characteristic feature for practicing the second embodiment. FIG. 9A is a view when the piston is seen from an air transfer tube 44. FIG. 9B is a sectional view of the piston.

The piston 40 is mainly constituted by three components including an air compressing portion 40a, a drive portion 40b, and a spring 40c as a biasing unit in this case, the piston proposed in this embodiment greatly differs from the conventional piston 40 in that a hole is formed in a central portion of the air compressing portion 40a. This hole serves as an air path extending from the side of the piston 40 on which air in the cylinder 43 is compressed to the rear side of the piston 40 which leads to the outside of the cylinder 43. Another important point of this structure is that as the distance between the drive portion 40b and another component decreases, the hole is sealed.

This embodiment has a structure in which this hole can be easily sealed by the tapered structure of the convex portion of the drive portion 40b and a rubber ring 40d, as shown in FIG. 9B. The tapered structure and rubber ring function as a drive valve which opens and closes the above air path.

Furthermore, a spring 40c is disposed to keep the air compressing portion 40a and the drive portion 40b at a predetermined distance L. Both the air compressing portion 40a and the drive portion 40b are guided by guide members (not shown), and hence can move only in the biasing axis direction of the spring 40c.

In a state in which the biasing force of the spring 40c keeps the spring 40c and the air compressing portion 40a at the predetermined distance L, the air path extending from the hole formed in the center of the air compressing portion 40a to the rear side of the piston is ensured, as indicated by the dotted line arrow in FIG. 9B. In a natural state, the air compressing portion 40a is separated from the drive portion 40b by the biasing force of the spring 40c, and the air path is ensured.

Assume that a force is applied to the spring 40c in a direction to change the distance between the drive portion 40b and the air compressing portion 40a to L1 (<L). In this case, letting k be the spring coefficient of the spring 40c, the elastic force of the spring 40c is given by k×(L−L1), and the area of the air path decreases due to the tapered structure of the convex portion of the drive portion 40b.

As the distance between the two components, i.e., the air compressing portion 40a and the drive portion 40b, decreases with an increase in force applied to the spring 40c, the convex portion provided on the drive portion 40b seals the hole of the air compressing portion 40a in the case of L1=0. As a result, the air path is sealed. In addition, the elastic force of the spring 40c in the sealed state is given by k×L.

In this case, the diameter of the hole formed in the air compressing portion 40a is designed to be sufficiently small as compared with the piston diameter so as to satisfy the function (to be described later).

Although this embodiment uses the spring as a biasing unit for the sake of ease of explanation, the embodiment may use another unit as long as it is a biasing unit having an equivalent function. This biasing unit forms a drive valve operating unit which opens and closes the air path by operating the drive valve. A force higher than the biasing force of the biasing unit of the piston 40 is applied to the drive valve when the position of the piston 40 in the cylinder 43 is set at a predetermined position or the moving velocity of the piston 40 becomes equal to or more than a predetermined velocity. As a result, the drive valve operates to close the air path. The biasing unit applies this biasing force to the drive valve in a direction to open the air path.

The devices described in the second embodiment are the same as those in the first embodiment except for the above piston structure, and hence a description of the arrangement and principle of each device and measurement procedures will be omitted.

The following will describe, with reference to FIGS. 10A to 12, how a pressure signal and a corneal shape changing signal change due to a piston shape as a characteristic feature when the same control operation as that in the first embodiment is performed.

FIGS. 10A to 10D show the positions and states of the piston at the time of solenoid control in this embodiment. FIG. 10A shows the initial position of the piston.

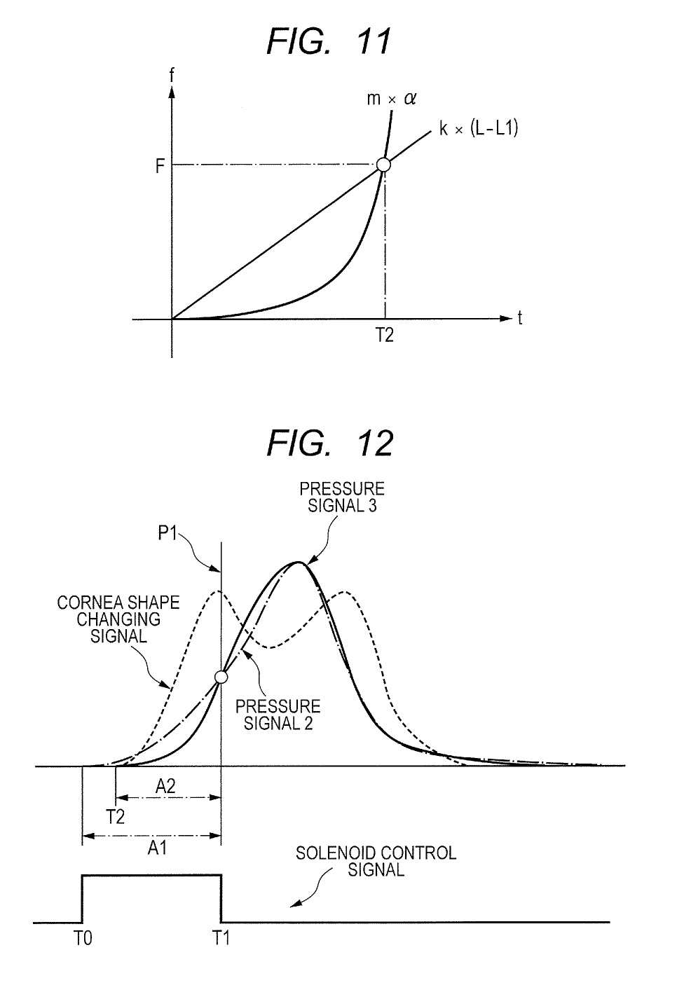

As energization to the solenoid starts in this state, a force F of the solenoid is applied to the drive portion 40b to accelerate the piston. In this case, FIG. 11 shows the relationship between the spring force and the force applied to the air compressing portion 40a due to the acceleration of the solenoid. The abscissa represents time t, and the ordinate represents a force f. Letting m be the mass of the air compressing portion 40a, when the spring 40c is pushed by the force of an acceleration a, the force represented by m×a acts on the air compressing portion 40a in a direction to push and compress the spring 40c due to friction in the piston and the law of inertia. Since the force m×a produced by the solenoid is much larger than the spring force k×(L−L1), the distance L1 between the two components, namely the drive portion 40b and the air compressing portion 40a, decreases, and the air path is closed at the instant when m×a=k×L, i.e., L1=0, as shown in FIG. 10B. A further description will be made, with the time of this instant being represented by T2.

FIG. 12 shows the relationship between a solenoid control signal, a pressure signal in the air chamber 23 in a case of using the piston in this embodiment, and a corneal shape changing signal corresponding to the eye E. Referring to FIG. 12, the abscissa represents the time from the measurement start time, and the ordinate represents the level of each signal. As in FIG. 5, the dotted line represents the corneal shape changing signal, and the solid line represents the pressure signal (pressure signal 3). For comparison, the chain line represents a pressure signal (pressure signal 2) in the control method according to the first embodiment.

In addition, for the sake of ease of explanation, the drive time based on a solenoid control signal coincides with the ON/OFF timing.

As described above, in the time interval from an initial state T0 to T2 described above, the internal pressure of the cylinder 43 does not rise even if energization to a solenoid 42 starts, due to the structure configured to discharge air through the air path formed in the piston 40.

The piston 40 therefore keeps accelerating without any air friction to start compressing air immediate after T2.

For this reason, the initial velocity of the piston at the start of compression in the second embodiment is higher than that in the first embodiment, and the gradient of the pressure signal is large. The time interval from the start of detection of a pressure signal to a point P1 becomes a time interval A2 (T1−T2) relative to A1 (T1−T0) in the related art. This makes it possible to obtain a desired pressure in a shorter period of time.

As described above, it is possible to obtain a synergetic effect by practicing the first embodiment with the structure of the piston 40 proposed in the second embodiment. It is possible to shorten the time period B1 in FIG. 5 in the first embodiment and the time period A1 in FIG. 5 in the second embodiment.

In addition, using the shape of the piston 40 described in the second embodiment can obtain further merits. FIG. 10C shows the position of the piston 40 after the end of measurement.

A general piston shape has the problem that when the piston in this state returns to the initial position in piston drive operation, the internal pressure of the cylinder 43 decreases to make the nozzle 22 draw tears from the eye to be inspected and dust and like in the air. In contrast to this, with the piston shape proposed in the second embodiment, since the direction of a force F′ of the return spring of the solenoid 42 coincides with that of the elastic force k×L of the spring 40c, a force acts in a direction to open the air path of the piston 40 as shown in FIG. 10D. When the air path opens, since the internal pressure of the cylinder 43 does not decrease even when the piston returns to the initial position, the nozzle 22 does not draw any tears and dust and the like.

### Other Embodiment

The present invention can be implemented by executing the following processing. That is, this processing includes supplying software (program) for implementing each function of the embodiments described above to a system or apparatus via a network or various types of recording media, and making the computer (or a CPU or MPU) of the system or apparatus read out and execute the program.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application Nos. 2012-130586, filed Jun. 8, 2012, and 2013-073218, filed Mar. 29, 2013, which are hereby incorporated by reference herein in their entirety.

What is claimed is:

1. A contactless tonometer including a corneal shape changing unit configured to change a shape of a cornea of an eye to be inspected by compressing air in a cylinder by using a piston which is disposed in the cylinder and operates from a movement start position, and puffing the compressed air from inside of the cylinder to the cornea, a piston control unit configured to control operation of the piston, and an intraocular pressure measurement unit configured to measure an intraocular pressure of the eye by detecting a state of a changed shape of the cornea,
comprising a piston volume changing unit configured to change an initial volume when the piston compresses the air in the cylinder.

2. A tonometer according to claim 1, wherein the piston volume changing unit changes the movement start position of the piston.

3. A tonometer according to claim 1, further comprising a piston position detection unit configured to detect a position of the piston,
wherein the piston control unit moves the piston to the movement start position and holds the piston at the movement start position based on a detection result obtained by the piston position detection unit.

4. A tonometer according to claim 3, wherein the piston is operated by a solenoid, and the piston control unit controls the piston by variable control and ON/OFF control of a drive current to the solenoid.

5. A tonometer according to claim 1, wherein the piston volume changing unit changes the initial volume in accordance with an intraocular pressure of the eye which is measured by the intraocular pressure measurement unit.

6. A tonometer according to claim 5, wherein the piston volume changing unit increases the initial volume when the intraocular pressure of the eye which is measured by the intraocular pressure measurement unit is not less than a predetermined value.

7. A tonometer according to claim 5, wherein the piston volume changing unit changes the initial volume in accordance with an intraocular pressure obtained by adding a predetermined value to the intraocular pressure of the eye which is measured by the intraocular pressure measurement unit.

8. A tonometer according to claim 6, further comprising a determination unit configured to determine whether the intraocular pressure of the eye which is measured by the intraocular pressure measurement unit is not less than a predetermined value for each measurement by the intraocular pressure measurement unit.

9. A tonometer according to claim 5, wherein the piston volume changing unit decreases the initial volume when the intraocular pressure of the eye which is measured by the intraocular pressure measurement unit is not more than a predetermined value.

10. A tonometer according to claim 1, wherein the piston includes an air path extending from a side in the cylinder on which air is compressed to an outside of the cylinder, a drive valve configured to open and close the air path, and a drive valve operating unit configured to operate the drive valve and closes the air path as a moving velocity of the piston becomes not less than a predetermined velocity.

11. A tonometer according to claim 10, wherein the drive valve operating unit includes a biasing unit configured to apply biasing force to the drive valve in a direction to open the air path, and
the biasing unit causes the piston to start compressing the air in the cylinder by closing the air path when a force applied to the piston becomes not less than the biasing force to cause the piston to compress the air.
